(19) United States
(12) Patent Application Publication
(10) Pub. No.: US 2012/0208780 A1
(43) Pub. Date: Aug. 16, 2012

(54) HYBRID NANOMATERIALS CONSISTING OF PSEUDOROTAXANES, PSEUDOPOLYROTAXANES, ROTAXANES, POLYROTAXANES, NANOPARTICLES AND QUANTUM DOTS

(75) Inventors: Mohsen Adeli, Khorramabad (IR); Mahdieh Kalantari, Khorramabad (IR); Maasoomeh Sagvand, Khorramabad (IR)

(73) Assignee: LORESTAN UNIVERSITY, Khorramabad - Lorestan (IR)

(21) Appl. No.: 13/028,936

(22) Filed: Feb. 16, 2011

(51) Int. Cl. A61K 31/715; C12Q 1/02; A61P 31/00; A61P 35/00; A61K 31/704; A61K 47/26; A61P 33/06; B82Y 30/00

(52) U.S. Cl. 514/34; 514/777; 514/58; 435/29; 977/773; 977/774

(57) ABSTRACT

This invention provides the synthesis of biocompatible and high functional hybrid nanomaterials consisting of pseudorotaxanes, pseudopolyrotaxanes, rotaxanes, polyrotaxanes, nanoparticles and quantum dots (QDs). The molecular self-assembly of hybrid nanomaterials lead to the formation of nano-objects with different shapes such as core-shell, spindle-like or necklaces. Due to their well-defined molecular self-assemblies, carbohydrate backbone, high functionality and several types of functional groups together with the high luminescence yield, thermal and physical properties and synthesized hybrid nanostructures were recognized as promising candidates for a wide range of applications.

### FIELD OF THE INVENTION

The present invention relates to the synthesis of hybrid nanomaterials based on pseudorotaxanes, pseudopolyrotaxanes, rotaxanes, polyrotaxanes, quantum dots and nanoparticles. The hybrid nanomaterials of the present invention have the collection of desirable properties of the individual nanomaterials.

### BACKGROUND OF THE INVENTION

Interlocked molecules such as rotaxanes, catenanes, molecular knots, and molecular necklaces have received much attention due to their potential application in molecular scale functional devices and machines [1-3]. Rotaxanes are macromolecules consisting of one or more rings and one or more axes, in which the dissociation of ring from axis is hindered by bulky groups (so-called stoppers) at both ends of the axis [4, 5]. There is no chemical bonding between rings and axis, and they are only interlocked mechanically [6]. Many cyclic components, such as calix[n]arenes [7-9], Crown ethers [10, 11], cyclodextrins [12-14], cucurbituril [15, 16], and cyclophanes [17] have been extensively used as ring for the construction of rotaxanes [18].

On the other hand, quantum dots, magnetic nanoparticles, metallic nanoparticles and other nanoparticles have been widely studied due to unique physical, chemical, optical and electronic properties. To date, various nanoparticles have been synthesized for a wide range of applications.

As such, homogenous thiolate gold nanoparticles coupled to biomolecules, such as DNA or proteins [19, 20], hold great promise for electron microscopy [21], nanoscale construction [22] and enzyme enhancement [23, 24]. In another example, tumor targeting chitosan nanoparticles have been introduced for optical/Magnetic resonance (MR) dual imaging [25].

Semiconductor quantum dots (QDs) as new classes of fluorophores have been widely used in solar cells [26], light emitting diodes [27, 28], laser technologies [29, 30], chemical sensing [31-33] and bio-imaging [34-38]. The general structure of QDs which are often used combined from an inorganic core, an inorganic shell, an organic shell.

Although rotaxanes, quantum dots and nanoparticles are extensively used in different fields, their applications in some promising fields are restricted by some of their disadvantages.

In order to improve the properties of single nanomaterials for different applications, their structures are modified by different modifier molecules. When the modifier is another nanomaterial or nanostructure, this strategy leads to “hybrid nanostructures”. Hybrid nanostructures are very young and promising systems in which several nanomaterials are combined or aggregated through predesigned strategies. This is a promising way to overcome the disadvantages of single nanomaterials and preparation of new nanostructures or nanodevices with desirable properties, because their cumulative properties are the result of properties of individual nanomaterials.

On the basis of the motivations described above, in this invention hybrid nanomaterials consisting of pseudorotaxanes, pseudopolyrotaxanes, rotaxanes, polyrotaxanes, nanoparticles and quantum dots were synthesized.

### SUMMARY OF THE INVENTION

The present invention relates to the synthesis of hybrid nanomaterials consisting of several building blocks, wherein said building blocks are pseudorotaxanes, pseudopolyrotaxanes, rotaxanes, polyrotaxanes, quantum dots, and nanoparticles. Covalent and non-covalent interactions between these building blocks lead to new nanostructures having a hybrid of properties of all individual nanomaterials. According to an exemplary embodiment of the present invention, a hybrid nanostructure consisting of a cyclodextrin-polyrotaxane, end-capped by cadmium selenide quantum dots linked to anticancer drugs was synthesized.

As mentioned synthesized hybrid nanomaterials are consisting of several nanomaterials, therefore they have a collection of their properties. For example when QDs are used as stoppers for polyrotaxanes, they not only hinder disassociation of rings from axes but also offer the properties of semiconductors in polyrotaxane structures. In addition to their individual properties new properties appear upon conjugating nanomaterials together. For example polyrotaxanes containing nanoparticles stoppers make new nano-objects through molecular self-assembly. Due to their multi-functionality and versatility of their structures, they could be used for different applications and proposes.

The present invention is presented herein according to the following preferred embodiments: 1. A hybrid nanomaterial comprising two or more building blocks selected from the group consisting of a rotaxane, a polyrotaxane, a pseudorotaxane, a pseudopolyrotaxane, a quantum dot, a polymer and a nanoparticle or any combination thereof.

Additionally or alternatively to 1, any device based on hybrid nanomaterials comprising two or more building blocks selected from the group consisting of a rotaxane, a polyrotaxane, a pseudorotaxane, a pseudopolyrotaxane, a quantum dot, a polymer and a nanoparticle or any combination thereof.

Additionally or alternatively to 1 or 2, any molecular self-assembly in which building blocks are hybrid nanomaterials comprising two or more building blocks selected from the group consisting of a rotaxane, a polyrotaxane, a pseudorotaxane, a pseudopolyrotaxane, a quantum dot, a polymer and a nanoparticle or any combination thereof.

2. The hybrid nanomaterial according to 1, comprising one quantum dot and at least one other building block selected from the group consisting of polyrotaxane, rotaxane, pseudopolyrotaxane and pseudorotaxane.

3. The hybrid nanomaterial according to 1, wherein the polyrotaxane, rotaxane, pseudopolyrotaxane or pseudorotaxane comprise any polymer or macromolecule as an axis and any molecule or macromolecule as a ring.

4. The hybrid nanomaterial according to 1, comprising one nanoparticle and at least one other building block selected from the group consisting of polyrotaxane, rotaxane, pseudopolyrotaxane and pseudorotaxane.

5. The hybrid nanomaterial according to any of 1-4, wherein the building blocks are connected via covalent interactions.

6. The hybrid nanomaterial according to any of 1-5, wherein the building blocks are connected via non-covalent interactions.

7. The hybrid nanomaterial according to any of 1-6, wherein the building blocks are connected via covalent and/or non-covalent interactions or any combination thereof.

8. The hybrid nanomaterial according to 6 or 7, wherein said non-covalent interaction comprises host-guest interaction, hydrogen bond, van der Waals interaction, electrostatic interaction, dispersion interaction, or any combination thereof.

9. The hybrid nanoparticle according to any of 1-8, comprising shapes selected from the group consisting of core-shell, spindle, spindle-like and necklace.

10. The hybrid nanomaterial according to any of 1-9, further comprising an end-capping agent.

11. The hybrid nanoparticle according to any of 1-9, further comprising an end-capping agent selected from the group consisting of beta-cyclodextrin, alpha-cyclodextrin, mercaptoacetic acid, a cysteine-comprising capping agent, a quantum dot and a cadmium selenide comprising quantum dot.

12. The hybrid nanomaterial according to any of 1-11, wherein the rotaxane, polyrotaxane, pseudorotaxane, and/or pseudopolyrotaxane comprises cyclodextrin.

13. The hybrid nanoparticle according to any of 1-12, wherein the rotaxane, polyrotaxane, pseudorotaxane, and/or pseudopolyrotaxane comprises a polymer containing a carbohydrate backbone, in particular a biocompatible carbohydrate backbone, more in particular polyethylene glycol.

14. The hybrid nanoparticle according to any of 1-13, comprising a cyclodextrin-polyrotaxane end-capped by quantum dots with a cysteine-comprising capping agent, a cyclodextrin-polyrotaxane end-capped by cadmium selenide quantum dots, a cyclodextrin-polyrotaxane end-capped by quantum dots with a beta-cyclodextrin and/or mercaptoacetic acid capping agent, a cyclodextrin-polyrotaxane end-capped quantum dot having covalent interactions between pseudopolyrotaxane and cadmium selenide quantum dots with a cysteine end-capping agent and/or a cyclodextrin-polyrotaxane end-capped quantum dot having non-covalent interactions between pseudopolyrotaxane and cadmium selenide quantum dots with a beta-cyclodextrin and/or a mercaptoacetic acid end-capping agents.

15. The hybrid nanoparticle according to any of 1-14, conjugated with an active compound.

16. The hybrid nanoparticle according to any of 1-14, conjugated with an active compound selected from a drug, preferably a prophylactic agent against malaria, an antibiotic or an anticancer drug such as doxorubicin or cis-diamminedichloroplatinum; or a vitamin, preferably folic acid.

17. The hybrid nanoparticle according to any of 1-16, wherein the active compound is conjugated through functional hydroxyl groups.

18. A drug delivery or drug targeting system comprising the hybrid nanomaterial according to any of 1-17 conjugated with an active compound, in particular a drug.

19. A diagnostic system comprising the hybrid nanomaterial according to any of 1-17.

20. A sensor or biosensor comprising the hybrid nanomaterial according to any of 1-17.

21. A nanocomposite comprising the hybrid nanomaterial according to any of 1-17.

22. A solar cell comprising the hybrid nanomaterial according to any of 1-17.

23. A biomolecular or cellular imaging system comprising the hybrid nanomaterial according to any of 1-17.

24. A method for the synthesis of a hybrid nanomaterial comprising the steps of conjugating a cysteine-cadmium comprising quantum dot through a nucleophylic reaction between functional amino groups thereof with end functional groups of a (pseudo)polyrotaxane.

25. The method according to 24, further comprising of conjugating carboxylate groups of the quantum dot with a drug to obtain a drug delivery system.

26. The method according to 24 or 25, wherein the drug comprises a prophylactic agent against malaria, an antibiotic or an anticancer drug, in particular doxorubicin or cis-diamminedichloroplatinum.

27. A method of delivering a hybrid nanomaterial according to any of 1-17, comprising of contacting cells with the hybrid nanomaterial for a time period sufficient to allow uptake of the hybrid nanomaterial.

28. The method according to 27, wherein the cell is contacted in vivo or in vitro with the nanomaterial.

29. A method for delivering an active compound or drug to a cell comprising of providing a hybrid nanomaterial according to any of 15-17 comprising of contacting a cell with the drug- or active compound-comprising hybrid nanomaterial.

30. The method according to 29, wherein the cell is contacted in vivo or in vitro.

31. The method according to 29 or 30, wherein the drug is a prophylactic agent against malaria, an antibiotic or an anticancer drug, such as doxorubicin or cis-diamminedichloroplatinum.

### BRIEF DESCRIPTION OF THE FIGURES

FIG. 1. Schematic representation of the synthesis of functionalized poly(ethylene glycol) (Cl-PEG-C1) (1), pseudopolyrotaxane (Ps-PR) (2), cyclodextrin-polyrotaxane end-capped by quantum dots with cysteine capping agent (PR-Cys-CdSe QDs) (3) and conjugation of doxorubicin (DOX) to PR-Cys-CdSe QDs (DOX-PR-Cys-CdSe QDs) (4).

FIG. 2. Schematic representation of the synthesis of cyclodextrin-polyrotaxane end-capped by quantum dots with beta-cyclodextrin (β-CD) and mercaptoacetic acid (MAA) capping agents (PR-CD/MAA-CdSe QDs) (a), conjugation of Cis-Diamminedichloroplatinum (CDDP) to PR-CD/MAA-CdSe QDs (CDDP-PR-CD/MAA-CdSe QDs) and conjugation of folic acid (FA) to CDDP-PR-CD/MAA-CdSe QDs (FA-CDDP-PR-CD/MAA-CdSe QDs) (b).

FIG. 3. XRD pattern of Ps-PR (a), quantum dots with cysteine capping agent (Cys-CdSe QDs) (b), PR-Cys-CdSe QDs (c) and DOX-PR-Cys-CdSe QDs (d).

FIG. 4. The UV-visible spectra of Cys-CdSe QDs (a), DOX (b), PR-Cys-CdSe QDs (c) and DOX-PR-Cys-CdSe QDs (d).

FIG. 5. UV-visible spectra of CDDP (a), FA (b), quantum dots with beta-cyclodextrin and mercaptoacetic acid capping agents (CD/MAA-CdSe QDs) (c), PR-CD/MAA-CdSe QDs (d), CDDP-PR-CD/MAA-CdSe QDs (e) and FA-CDDP-PR-CD/MAA-CdSe QDs (f).

FIG. 6. Photograph of water solutions of CD/MAA-CdSe QDs (I), PR-CD/MAA-CdSe QDs (II), CDDP-PR-CD/MAA-CdSe QDs (III) and FA-CDDP-PR-CD/MAA-CdSe QDs (IV) under sunlight(A) and UV irradiation(B).

FIG. 7. Fluorescence image of CD/MAA-CdSe QDs (a) and PR-CD/MAA-CdSe QDs (b).

FIG. 8. Fluorescence image of Cys-CdSe QDs (a) and PR-Cys-CdSe QDs (b).

FIG. 9. Photoluminescence spectra of Cys-CdSe QDs (a), PR-Cys-CdSe QDs (b) and DOX-PR-Cys-CdSe QDs (c).

FIG. 10. Zeta potential values of Ps-PR (a), Cys-CdSe QDs (b), PR-Cys-CdSe QDs (c) and DOX-PR-Cys-CdSe QDs (d).

FIG. 11. DLS diagram of Ps-PR (a), Cys-CdSe QDs (b), PR-Cys-CdSe QDs (c) and DOX-PR-Cys-CdSe QDs (d).

FIG. 12. TEM images of Cys-CdSe QDs (a), PR-Cys-CdSe QDs (b and c) and proposed process for molecular self-assembly of PR-Cys-CdSe QDs on graphite holder (d).

FIG. 13. AFM images of Ps-PR, phase contrast (a), topology (b) and proposed process for molecular self-assembly of Ps-PR on glass holder (c).

FIG. 14. AFM images of PR-Cys-CdSe QDs, topology (a), phase contrast (b) and proposed process for molecular self-assembly of PR-Cys-CdSe QDs on glass holder (d).

FIG. 15. TEM images of CD/MAA-CdSe QDs (a) and PR-CD/MAA-CdSe QDs (b). Topology (c) and phase contrast (d) AFM images of Ps-PR self-assemblies. Topology (e and g) and phase contrast (f and h) AFM images of PR-CD/MAA-CdSe QDs self-assemblies. Spherical self-assemblies of PR-CD/MAA-CdSe QDs associated together linearly lead to rod-like objects. AFM images show the association of spherical self-assemblies (i) and SEM image of the final product of association of spherical self-assemblies (j).

FIG. 16. Changing the intensity of λₘₐₓ of ferrocene versus time upon transferring α-CDs rings from dialysis bag at different pHs (a). Proposed mechanism for releasing of α-CDs rings from PEG axes and transferring to external solution (b). Potential application of synthesized hybrid nanomaterials for simultaneously active and passive targeting of anticancer drugs to tumors (c).

FIG. 17. The MTT (3-(4,5-dimethylthiazol-2-yl)-2,5-diphenyltetrazolium bromide) assay results for Ps-PR, Cys-CdSe QDs, PR-Cys-CdSe QDs and DOX-PR-Cys-CdSe QDs containing (0.333-0.666 mg/ml) of DOX.

FIG. 18. Cell cycle assay results for control (a) and Ps-PR treated cells (b).

FIG. 19. Fluorescence microscopy images of Cys-CdSe QDs (a) and PR-Cys-CdSe QDs (b) treated cells after 1 h.

FIG. 20. The native gel electrophoresis of treated human transferrin.

Table 1. Percent quota of cell cycle phases for bare and coated SPION treated cells.

TABLE 1

| Sample | Sub G₀G₁ | G₀G₁ | S | G₂/M |
|---|---|---|---|---|
| Control | 10.67 | 65.61 | 13.04 | 10.68 |
| Ps-PR | 11.19 | 59.02 | 15.55 | 14.24 |

### DETAILED DESCRIPTION OF THE INVENTION

Herein after referring to the accompanying drawings, an embodiment of the present invention will be described; this should not be construed as limiting the scope of the present invention.

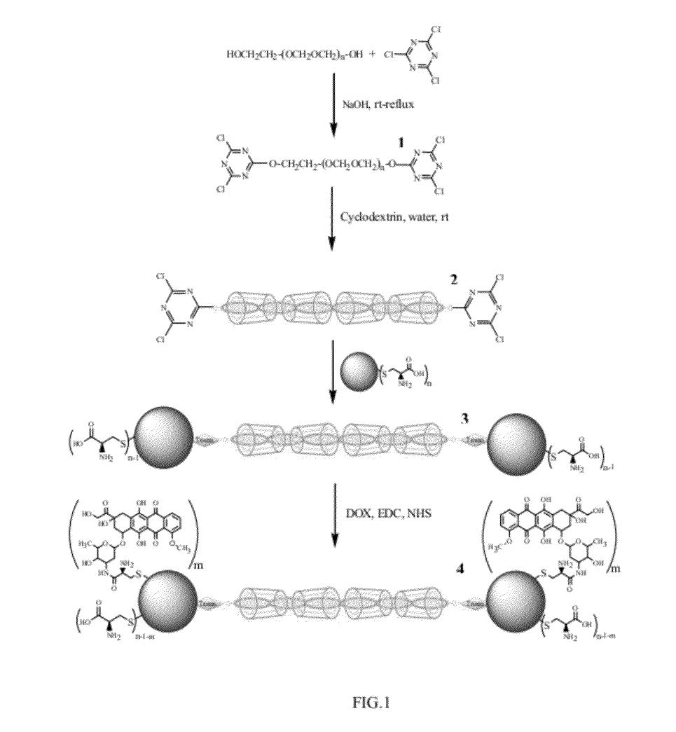

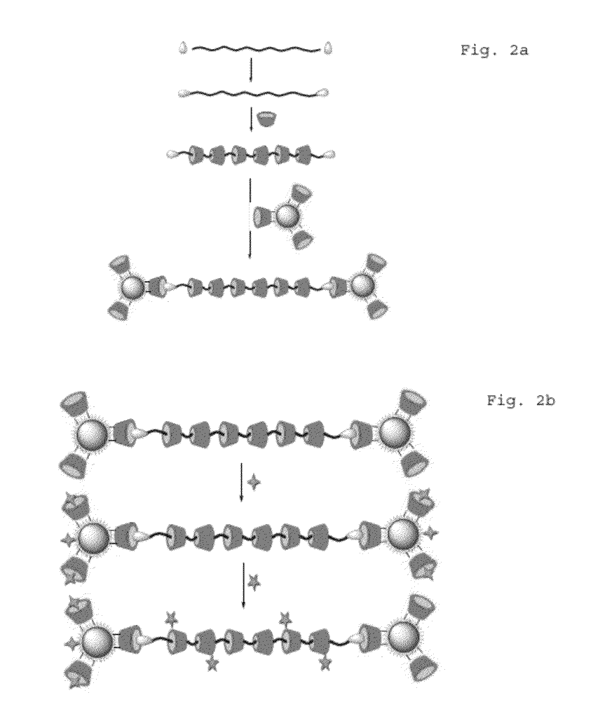

FIGS. 1 and 2 are representations schematically showing the method for synthesis of a hybrid nanomaterial according to an embodiment of the present invention. In this embodiment, the invention provides a process for forming a cyclodextrin-polyrotaxane, end-capped by cadmium selenide quantum dots. In these nanostructures, pseudopolyrotaxane (Ps-PR) consist of α-cyclodextrin (α-CD) rings and Polyethylene glycol (PEG) axis as biocompatible and high functional platforms have been capped by cadmium selenide quantum dots (CdSe QDs).

As shown in FIGS. 1 and 2, PEG containing end triazin groups was prepared first. Functionalization of PEG using reactive and hydrophobic triazin molecules not only increase the favor interactions between PEG and cavity of α-cyclodextrins, leading to pseudopolyrotaxanes in a short time, but also create reactive sites on the heads of pseudopolyrotaxanes, Ps-PR, to react with CdSe QDs and obtaining the polyrotaxane end-capped QDs. In the present embodiment, we prepared two types of cyclodextrin-polyrotaxane end-capped QDs:

i) cyclodextrin-polyrotaxane end-capped QDs based on covalent interactions between pseudopolyrotaxane (Ps-PR) and cadmium selenide quantum dots with cysteine capping agent.

ii) cyclodextrin-polyrotaxane end-capped QDs based on non-covalent interactions between pseudopolyrotaxane (Ps-PR) and cadmium selenide quantum dots with beta-cyclodextrin (β-CD) and mercaptoacetic acid (MAA) capping agents.

There are several key roles for CdSe QDs in these hybrid materials: (a) stopper, dissociation of α-cyclodextrin rings from PEG axis is hindered by bulky CdSe QDs (b) luminescence nanoprobe, for biomolecular and cellular imaging (c) bioconjugate platforms, attachment of Cis-Diamminedichloroplatinum (CDDP) and doxorubicin (DOX) as anticancer drugs to their surface functional groups.

As shown in FIG. 1, for synthesis of PR-Cys-CdSe QDs, Cys-CdSe QDs were conjugated to Ps-PR through nucleophilic reaction between amino functional groups of Cys-CdSe QDs and end functional groups of pseudopolyrotaxane. Then carboxylate groups of QDs were used for conjugating of DOX molecules to the PR-Cys-CdSe QDs and preparation of the DOX-PR-Cys-CdSe QDs as drug delivery system.

Another cyclodextrin-polyrotaxane, end-capped by CdSe QDs, PR-CD/MAA-CdSe QDs, were obtained via host-guest relationship between end triazine groups of pseudopolyrotaxane and beta-cyclodextrins conjugated onto the surface of CD/MAA-CdSe QDs. To prove the efficacy of synthesized polyrotaxanes as drug delivery and targeting systems, CDDP as an anticancer drug and folic acid (FA) as tumor-recognition module were conjugated to PR-CD/MAA-CdSe QDs to obtain CDDP-PR-CD/MAA-CdSe QDs and FA-CDDP-PR-CD/MAA-CdSe QDs, respectively (shown in FIG. 2).

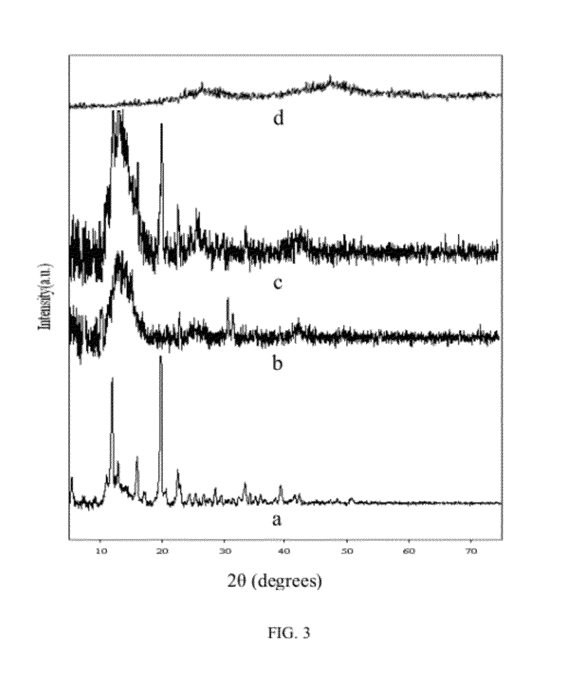

FIG. 3 shows XRD pattern of Ps-PR (a), Cys-CdSe QDs (b), PR-Cys-CdSe QDs (c) and DOX-PR-Cys-CdSe QDs (d). The XRD pattern of Ps-PR (FIG. 3a) with several peaks at 12.27, 17.25, 23.25 and the main one at 19.6° represents the channel-type crystalline structure. In Cys-CdSe QDs diffraction peaks at 2θ=25,42° are attributed to the (111) and (220) crystalline planes of cubic CdSe, respectively. PR-Cys-CdSe QDs (FIG. 3c) show main diffraction peaks of both the CdSe QDs and Ps-PR at almost 2θ=19.6, 25,42°, indicating that CdSe QDs and Ps-PR retain their crystalline structure in PR-Cys-CdSe QDs. The comparison of the FIG. 3d with FIGS. 3a and b reveals that the XRD pattern of DOX-PR-Cys-CdSe QDs is quite different from those for Cys-CdSe QDs and Ps-PR.

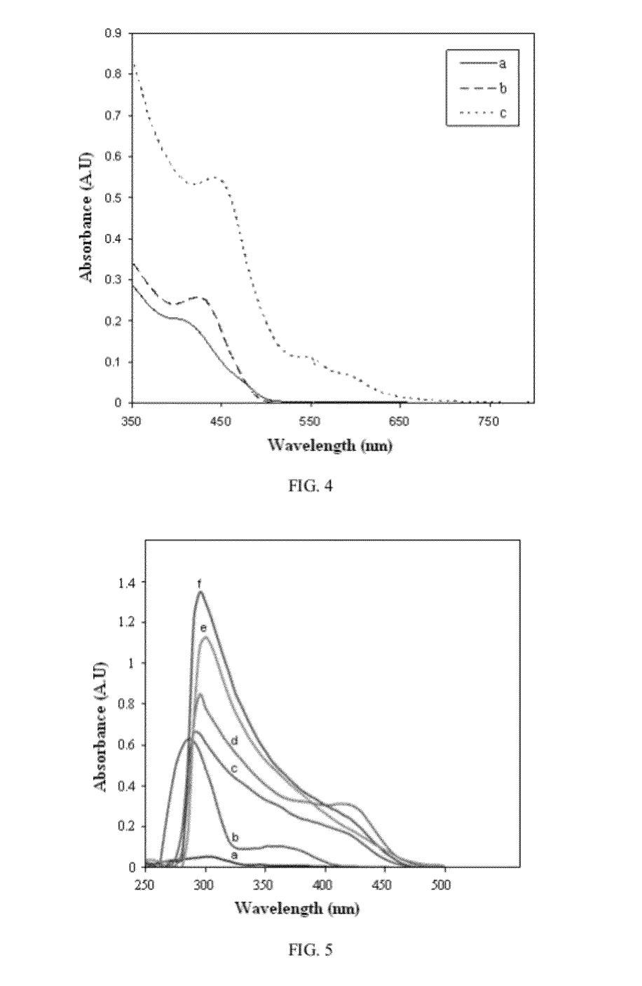

FIG. 4 shows the UV-visible spectra of Cys-CdSe QDs, DOX, PR-Cys-CdSe QDs and DOX-PR-Cys-CdSe QDs. The absorption spectrum of the Cys-CdSe QDs (FIG. 4a) displays an excitonic peak around 414 nm. The band gap (Eg) of Cys-CdSe QDs, calculated by E=hc/λ, formula, is 2.99 eV. Therefore, the absorption edge of Cys-CdSe QDs is blue-shifted as compared with the bulk CdSe (Eg=1.74 eV). A red shift for the maximum absorption wavelength (λₘₐₓ) of QDs upon conjugation to the Ps-PR is observed (FIG. 4c). The UV-visible spectrum of DOX-PR-Cys-CdSe QDs is quite different from those for DOX and PR-Cys-CdSe QDs and it shows a λₘₐₓ at 453 which is higher and lower than that for PR-Cys-CdSe QDs, 434 nm, and DOX, 496 nm, respectively.

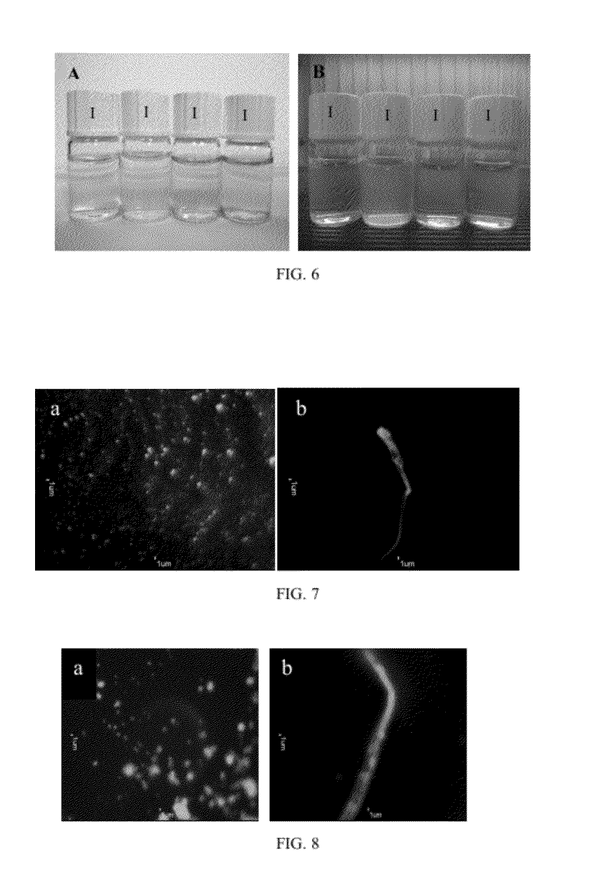

FIG. 5 shows the UV-visible spectra of CD/MAA-CdSe QDs, PR-CD/MAA-CdSe QDs, CDDP-PR-CD/MAA-CdSe QDs, FA-CDDP-PR-CD/MAA-CdSe QDs, CDDP and FA. The absorption spectrum of the CD/MAA-CdSe QDs displays Plasmon absorbance band centered on 415 nm (Eg=2.98 eV) and a shoulder at 362 nm. The Plasmon absorbance bands of PR-CD/MAA-CdSe QDs appeared at 420 nm with a slight red shift which is assigned to the formation of complex between the end triazine groups of pseudopolyrotaxane and beta-cyclodextrins conjugated onto the surface of CD/MAA-CdSe QDs. Due to the covering of the QDs upon conjugation of CDDP and FA to the PR-CD/MAA-CdSe QDs the Plasmon absorbance bands disappeared.

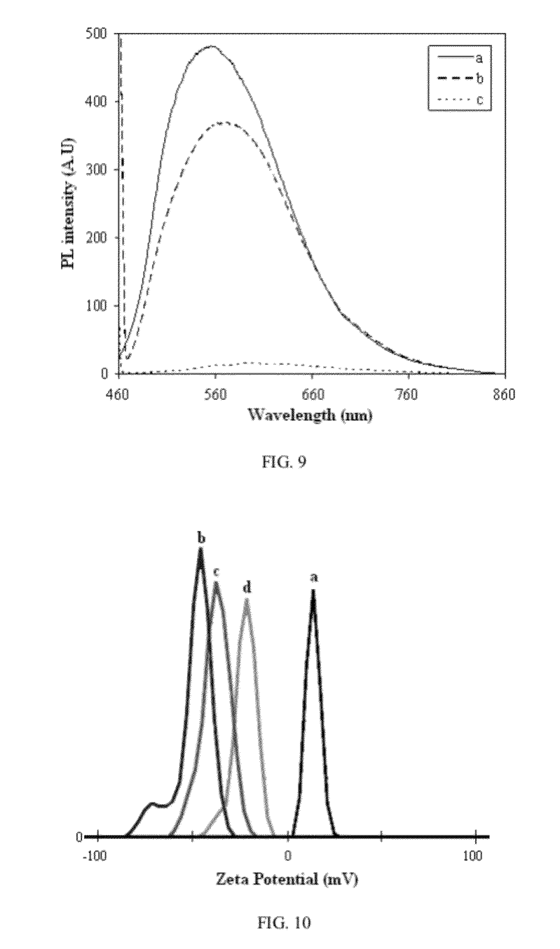

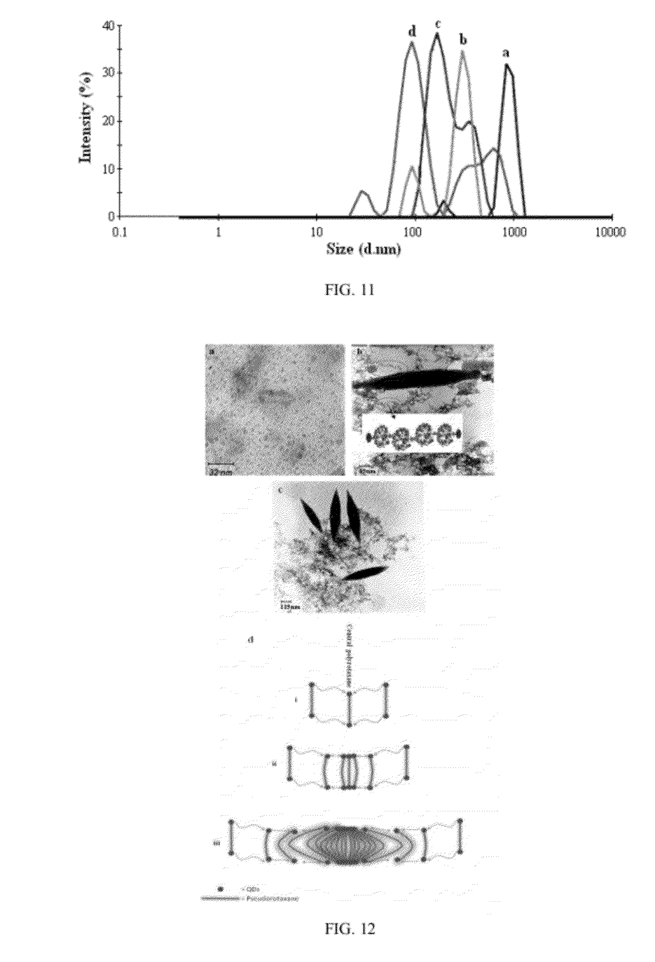

FIG. 6 shows the images of water solutions of CD/MAA-CdSe QDs, PR-CD/MAA-CdSe QDs, CDDP-PR-CD/MAA-CdSe QDs and FA-CDDP-PR-CD/MAA-CdSe QDs under sunlight (A) and UV irradiation (B). The fluorescence images of CD/MAA-CdSe QDs and PR-CD/MAA-CdSe QDs in the solid state are represented in FIG. 7. All samples had a good fluorescence emission (see FIGS. 6 and 7) and their color under UV irradiation was green. No precipitation or quenching was observed after several months.

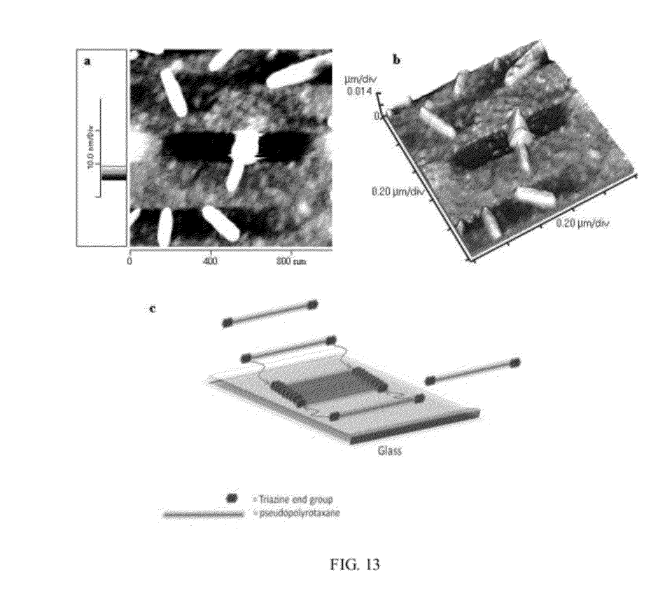

FIG. 8 shows the fluorescence images of Cys-CdSe QDs and PR-Cys-CdSe QDs in the solid state exited using a 550 nm radiation. The bright images clearly show that both samples have a good luminescence.

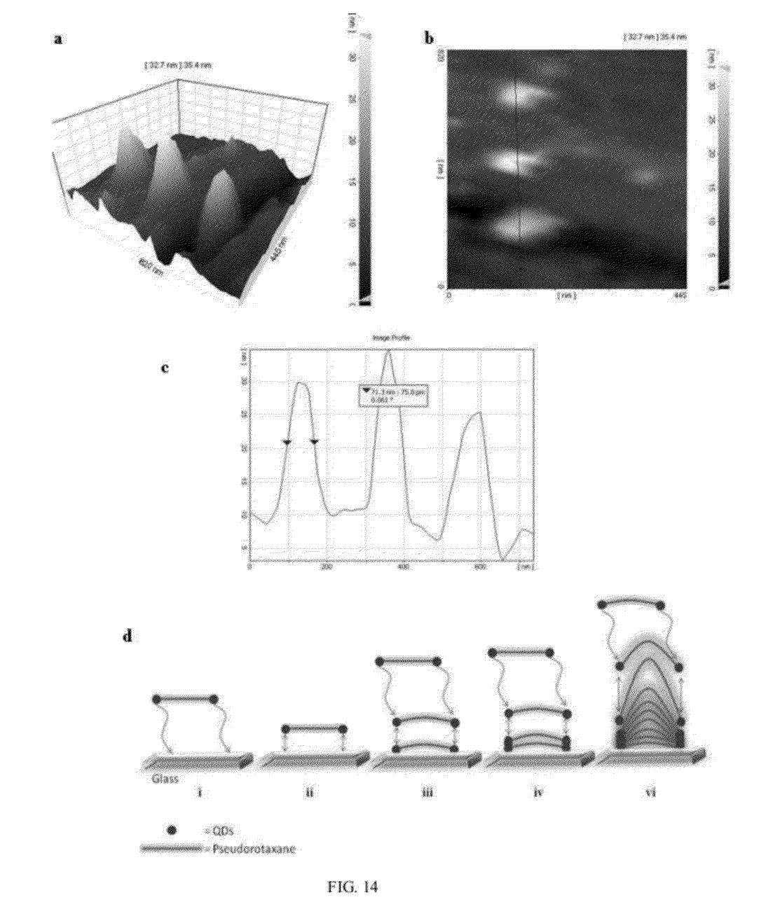

Photoluminescence measurement was carried out to investigate the effect of Ps-PR and DOX on the optical properties of Cys-CdSe QDs. FIG. 9 shows the Photoluminescence spectra of Cys-CdSe QDs, PR-Cys-CdSe QDs and DOX-PR-Cys-CdSe QDs. Cys-CdSe QDs and PR-Cys-CdSe QDs are highly luminescent both in solution and solid state, although a red shift for the maximum emission wavelength of Cys-CdSe QDs from 552 to 566 nm is observed upon conjugation of Ps-PR to them. Confirming the result of Photoluminescence experiments, conjugation of DOX molecules to PR-Cys-CdSe QDs quenches their luminescence which is assigned to overlap their emission and excitation wavelengths.

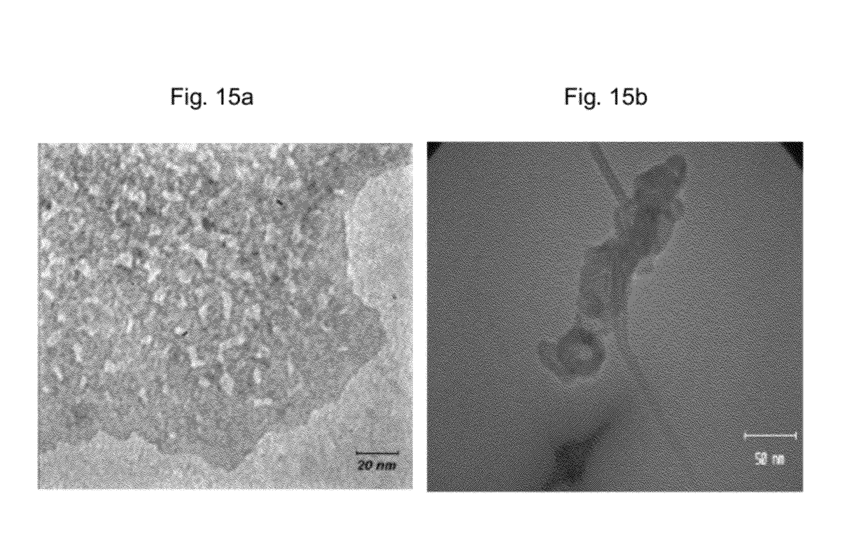

Zeta potential measurement was taken in water to obtain the information about surface charge of prepared samples and results are demonstrated in FIG. 10. The zeta potential measurements showed “+13” overall surface charge for Ps-PR assigned to the protonation of the nitrogen atoms of triazin groups. Cysteine isoelectric point (PI) is 5.07; therefore in the natural pH the surface charge of QDs with cysteine capping agent tends to negative values. However the surface charge for PR-Cys-CdSe QDs was “−37” which is the summation of the surface charge of QDs and Ps-PR. There are several types of functional groups in the structure of PR-Cys-CdSe QDs, amino and carboxyl functional groups onto the surface of QDs and hydroxyl functional groups of cyclodextrin rings, therefore they are able to transport several types of therapeutic or targeting agents simultaneously. In this work DOX molecules were conjugated to PR-Cys-CdSe QDs through reaction between carboxyl functional groups of QDs and amino functional groups of DOX molecules. The Zeta potential value for DOX-PR-CdSe QDs was “−22”. If each decreased negative charge unit for PR-CdSe QDs, after reaction with DOX molecules, assigned to the conjugation of one molecule DOX to carboxyl functional groups of PR-Cys-CdSe QDs, then the number of conjugated DOX molecules to PR-Cys-CdSe QDs can be estimated “15” roughly.

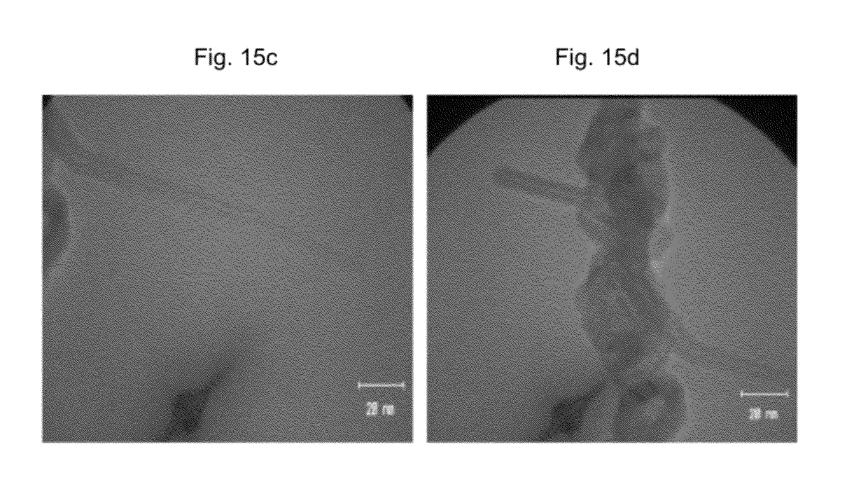

FIG. 11 shows DLS diagrams of Ps-PR, Cys-CdSe QDs, PR-Cys-CdSe QDs and DOX-PR-Cys-CdSe QDs. High functionality and polarity are two factors that encourage the synthesized hybrid nanostructures toward molecular self-assembly in the aqueous solutions. Different sizes for each object and appearance of the main peaks in the large size regions in DLS diagrams indicate that they are self-assembling in water at room temperature. The main driving force for molecular self-assembly of Cys-CdSe QDs in water is attraction between the negative and positive charges on their surfaces. However the big size of Ps-PR comes back to their poor solubility in water. Conjugation of Cys-CdSe QDs to Ps-PR not only decreases the surface charge of QDs and therefore decreases the attraction between their negative and positive charges but also increases the water solubility of Ps-PR. These two factors are the main reasons to decrease the size of PR-Cys-CdSe QDs in water in compare to that for QDs and Ps-PR. Conjugation of DOX molecules to PR-Cys-CdSe QDs decrease the size of their assemblies for the same reasons.

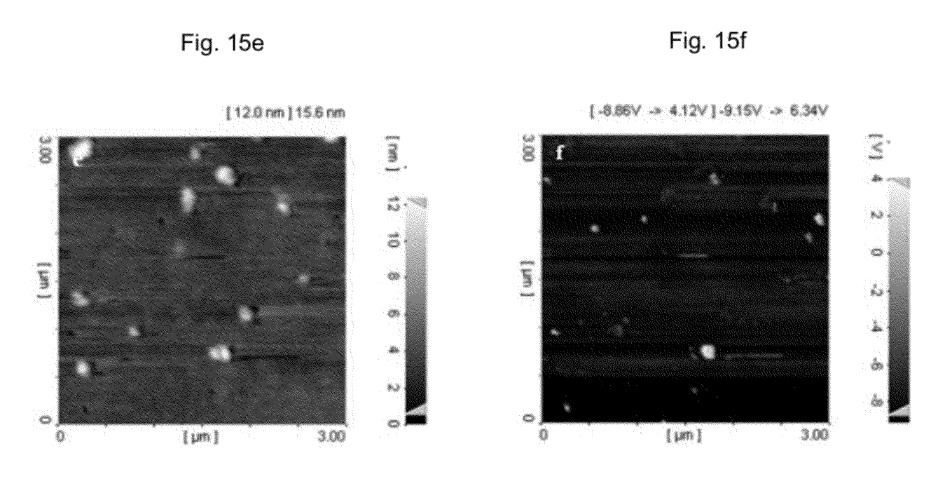

FIG. 12 shows the TEM images of Cys-CdSe QDs and PR-Cys-CdSe QDs. TEM images reveal Cys-CdSe QDs as spherical particles with an average size around 4 nm (FIG. 12a) and PR-Cys-CdSe QDs as necklace-like objects consist of QDs beads and Ps-PR linkages with a thickness around 2 nm, which is very close to the expected thickness for a single polyrotaxane consisting of PEG axes and α-cyclodextrin rings (FIG. 12b).

As can be seen in FIGS. 12b and 12c, polyrotaxanes are self-assembling to form spindle-like objects in which QDs are directed toward inside and a layer of Ps-PR is surrounding them. The thickness and length of molecular self-assemblies are around 50 and 300 nm, respectively. As it is evaluated by DLS experiments, electrostatic interactions between the functional groups of cysteine, force QDs to aggregate in the solution state strongly. Hence it seems the main driving force for the self-assembly of PR-Cys-CdSe QDs is the electrostatic interactions between end-capping QDs. In a proposed process for the molecular self-assembly of PR-Cys-CdSe QDs; first heads of a central PR-Cys-CdSe QDs interact with the heads of two neighbor PR-Cys-CdSe QDs through electrostatic interactions to create a central block, then Ps-PR backbones which are containing a large number of hydroxyl functional groups interact together non-covalently, for example through hydrogen bonding, and molecular self-assemblies growth. As aggregations are growing the backbone of PR-Cys-CdSe QDs should bend to have strong interactions through their heads. The growth and bending of PR-Cys-CdSe QDs is limited by the rigidity and limit length of their backbone leading to spindle-like self-assemblies.

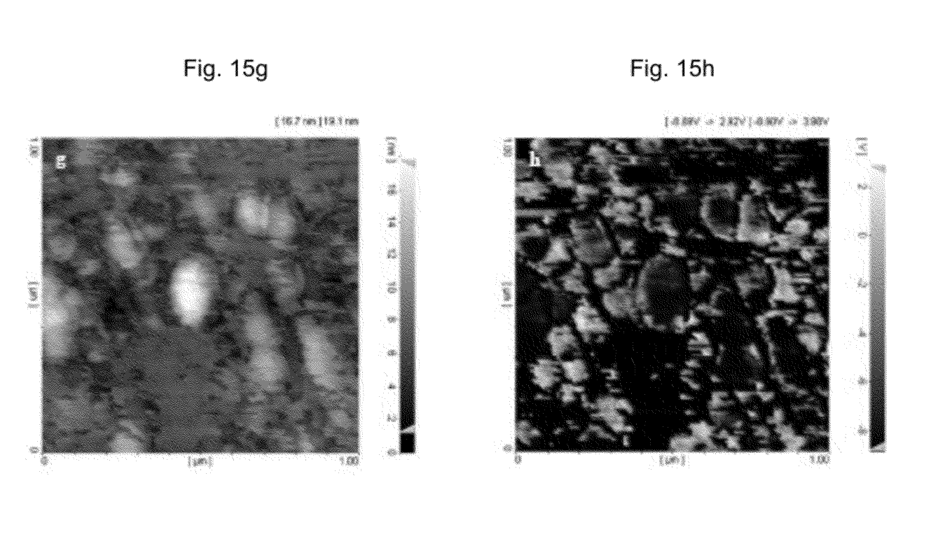

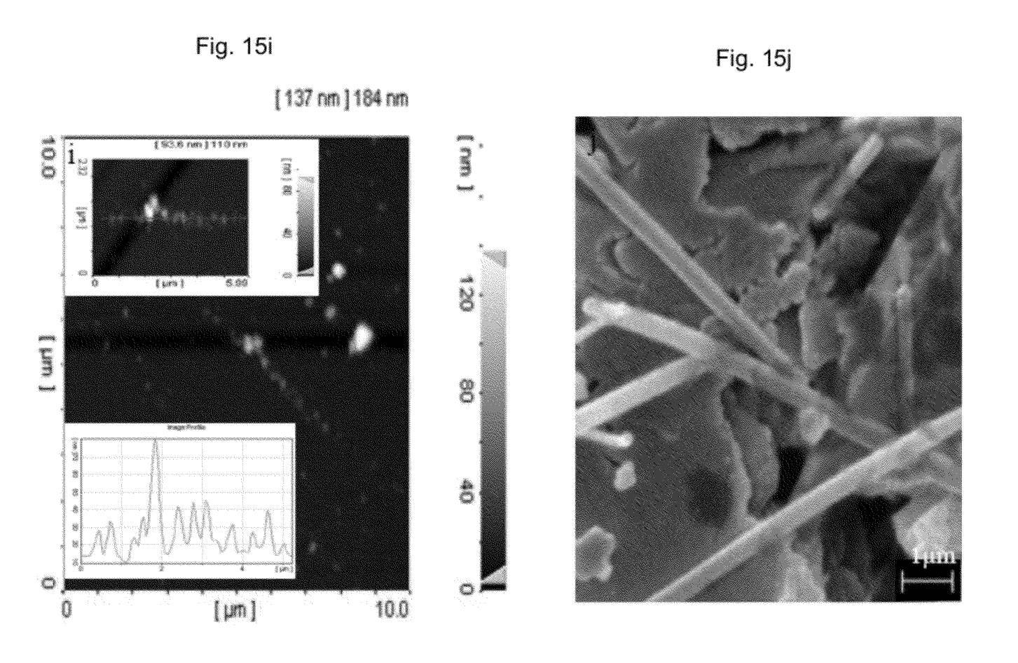

In order to clarify this proposed process and role of the head groups of PR-Cys-CdSe QDs in their molecular self-assembly, AFM images of Ps-PR and PR-Cys-CdSe QDs on glass holder were recorded. According to these images, Ps-PR were self-assembled as rod-like objects in the horizontal position and their length, width and height was around 250, 50 and 4 nm respectively, while PR-Cys-CdSe QDs formed the semi spindle-like molecular self-assemblies with a 70-100 nm width and 25-30 nm height. There are two reasons to prove the key role of QDs as the head groups of PR-Cys-CdSe QDs in their self-assembly using observed AFM images. The first reason is the difference between the shapes of molecular self-assemblies of PR-Cys-CdSe QDs recorded by AFM and TEM. Due to the interactions between the polar surface of glass and QDs, it plays the role of central PR-Cys-CdSe QDs in molecular self-assembly, therefore self-assemblies are half of spindle-like self-assemblies observed by TEM (FIG. 14a). The second reason is the difference between the shape of molecular self-assemblies of Ps-PR and PR-Cys-CdSe QDs. In spite of the head groups of PR-Cys-CdSe QDs, QDs, triazin groups in Ps-PR are hydrophobic and there is not a strong interaction between them and the glass surface but they can interact together horizontally to make rod-like self-assemblies (FIG. 13a). The phase contrast images of PR-Cys-CdSe QDs show that molecular self-assemblies are hybrid materials and contain dark points surrounded by white shells (FIG. 14b).

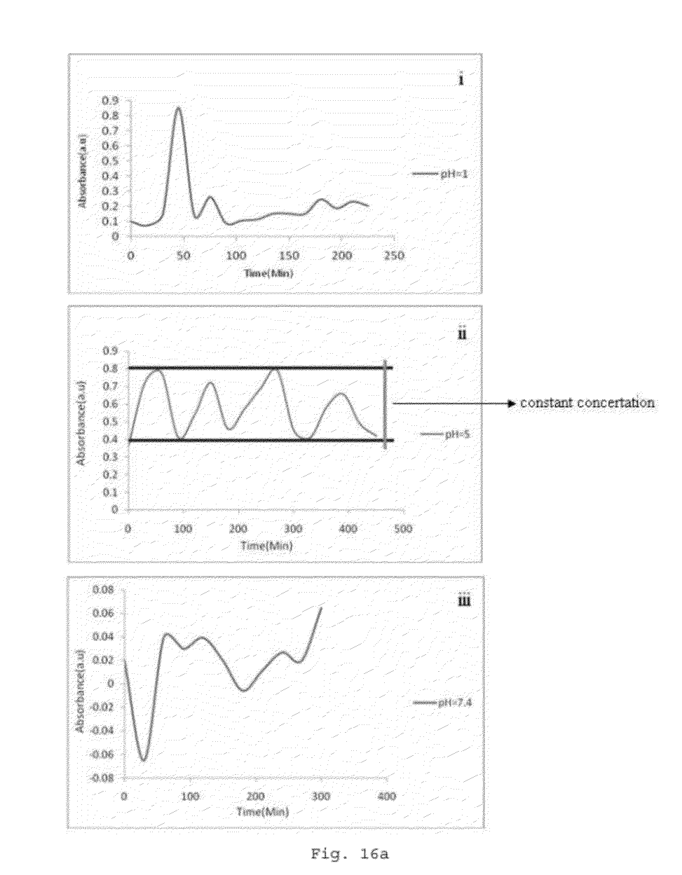

FIGS. 15a and b shows the TEM images of CD/MAA-CdSe QDs and PR-CD/MAA-CdSe QDs. Based on these images CD/MAA-CdSe QDs are not spherical in spite of those containing simple capping agents such as mercaptoacetic acid. They appeared as worm like objects with an average size around 10 nm, probably due to molecular self-assembly caused by beta-cyclodextrin capping agents.

Hydrophobic interactions between end triazine groups and interactions between hydroxyl groups of backbone in Ps-PR lead to molecular self-assemblies, of which their height, length and width are around 7, 200 and 50 nm, respectively (FIG. 15c and d). In fact QDs dominate the molecular self-assemblies of polyrotaxanes with QDs stoppers. FIGS. 15e and f show the topology and phase contrast, AFM images for self-assemblies of PR-CD/MAA-CdSe QDs in which sperical objects with an average size around 150 nm can be observed. Comparison of the topology and phase contrast images of PR-CD/MAA-CdSe QDs, especially in higher magnifications (FIG. 15g and h), show that they consist of two phases. This proves that in the molecular self-assemblies, Ps-PR and CD/MAA-CdSe QDs are associated together and are not independent.

It was found that primary self-assembly of PR-CD/MAA-CdSe QDs created spherical molecular self-assemblies which in turn were more associated together linearly and finally led to rod-like objects (FIGS. 15i and j).

Molecular self-assemblies are products of what is so called the “bottom-up” approach in nanotechnology. Due to the non-covalent interactions between their building blocks, one of the potential applications of the molecular self-assemblies is recognized in the drug delivery field, because they will degrade back into individual monomers that can be broken down by the in vivo environment. Recent studies in biodegradable polyrotaxanes focused on various stimuli-triggered responses such as enzymes, pH, redox and temperature. As mentioned before, PR-CD/MAA-CdSe QDs were synthesized based on a non-covalent interaction between pseudopolyrotaxane and CD/MAA-CdSe QDs. Herein all species are assembled by non-covalent interactions, therefore controlled disassociation of cyclodextrin rings from PEG axes through disturbing inclusion complexes between β-CD-graft-CdSe QDs and end triazine groups of Ps-PR lead to a control in the release of drug molecules conjugated to their hydroxyl functional groups. This could be achieved either by introducing a new guest molecule that can form inclusion complex with β-CD-graft-CdSe QDs with a higher affinity than triazine groups of Ps-PR or changing the pH, because host-guest relationship between β-CD-graft-CdSe QDs and end triazine groups of Ps-PR and also between PEG axes and α-CDs are pH sensitive.

To examine the first route, PR-CD/MAA-CdSe QDs was placed in a dialysis bag poured in a methanol solution of ferrocene. The intensity of UV-vis absorbance of the ferrocene solution was abruptly raised upon addition of pyrene to the dialysis bag showing disassociation of α-CDs rings from PEG axes and transferring from the dialysis bag to the external, ferrocene, solution through occupying all host sites on β-CD-graft-CdSe QDs by ferrocene molecules.

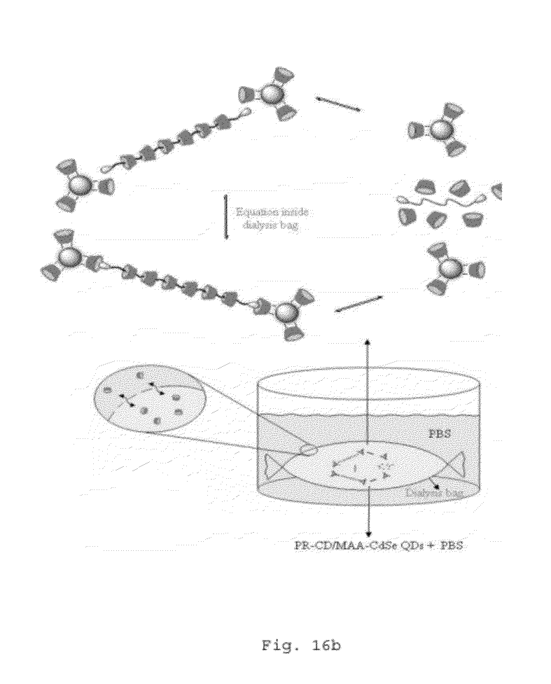

In order to investigate the release of α-CDs rings from PEG axes in different pHs, an aqueous solution of polyrotaxane was placed in a dialysis bag and it was transferred to a flask containing the same aqueous solution, external solution, then certain volumes of external solution were removed in interval times and added to a methanol solution of ferrocene and UV-vis spectra of a ferrocene was recorded. Results are shown in FIG. 16a (i, ii and iii for pH 1, 5 and 7 respectively). As it can be seen release of α-CDs rings from PEG axes is pH sensitive so that at pH 5 it is reversible while at pH 7 is less reversible and at pH 1 it is irreversible.

The concentration of α-CDs rings in the dialysis bag is high initially; therefore they transfer from the membrane to external solution leading to an increase in the concentration of α-CDs rings in the external solution and finally transferring them from membrane inversely (FIG. 16b).

Due to their molecular self-assemblies, size of PR-CD/MAA-CdSe QDs, CDDP-PR-CD/MAA-CdSe QDs and FA-CDDP-PR-CD/MAA-CdSe QDs in aqueous solutions is several hundred nm, which is an advantage for these systems to avoid nonspecific interactions and fast clearance from blood and therefore leading to long circulation upon administration. However they will cross the tissue endothelium barrier and introduce into the interstitial space of tissues slowly. Disassociation of stoppers and rings from polyrotaxanes will lead to cross barriers and introduce the cells through cell membranes quickly (FIG. 16c).

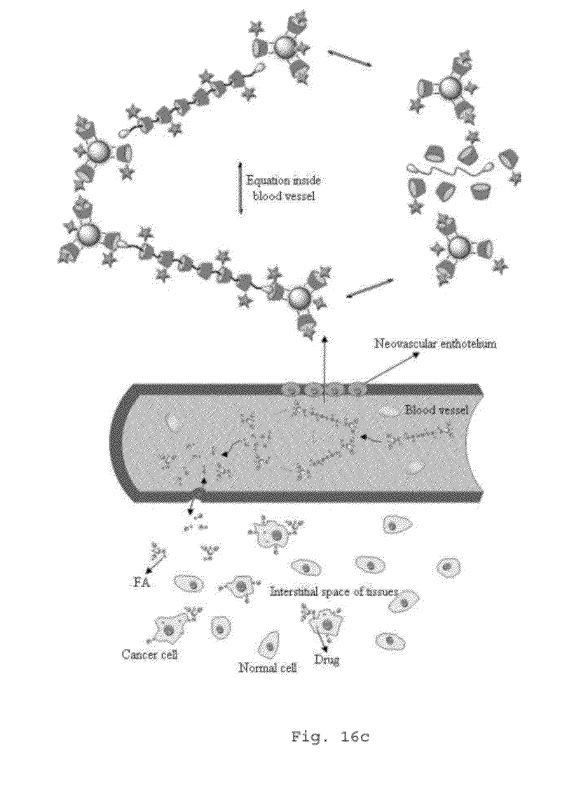

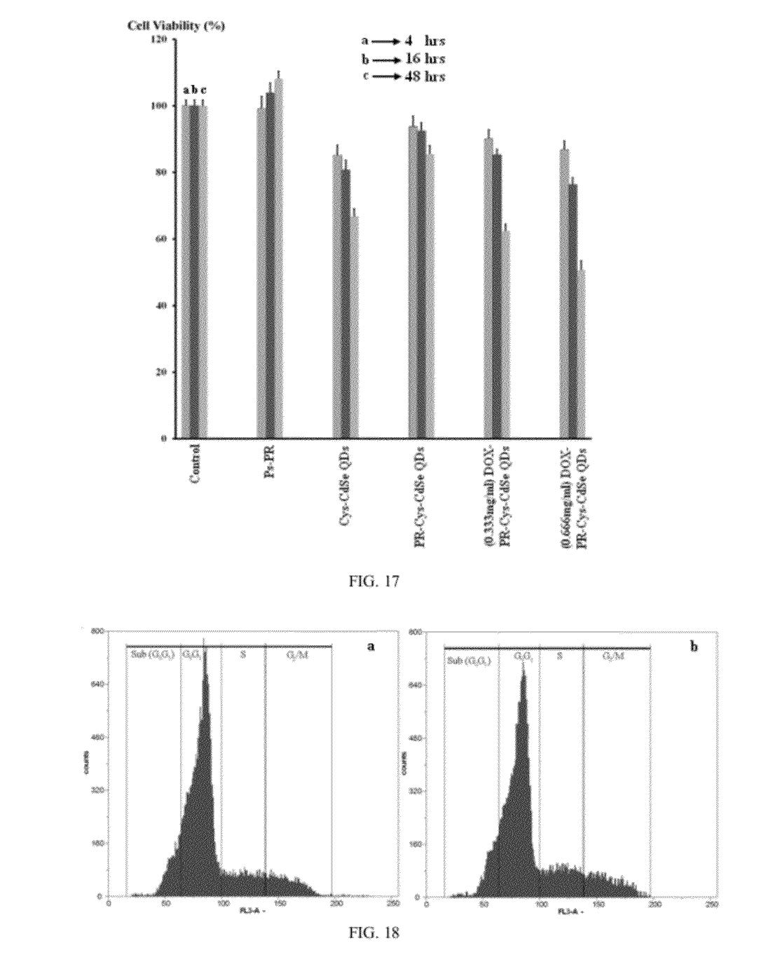

In order to investigate the potential application of hybrid nanostructures and their self-assemblies in nanomedicine and to understand their limitation and capability as nano-excipients in biological systems, short-term in vitro cytotoxicity tests were conducted on mouse tissue connective fibroblast adhesive cell line (L929). MTT results for Ps-PR, Cys-CdSe QDs, PR-Cys-CdSe QDs and DOX-PR-Cys-CdSe QDs are shown in FIG. 17. Modified MTT (3-(4,5-dimethylthiazol-2-yl)-2,5-diphenyltetrazolium bromide) assay showed not only any toxicity up to 1.6 mg/ml for Ps-PR but also in long incubation times, 16 and 48 h, an increase in the growth of incubated cells was observed against untreated control cells. According to our hypothesis, the positive effect of Ps-PR on the growth of the treated cells is related to their role in the metabolism of cells after transferring from the cell membrane. Due to the carbohydrate backbone of Ps-PR, it can be used as the source of energy by cells and therefore lead to an increase in the growth and division of the cells. In order to confirm this hypothesis, cell-cycle assay was performed for a L929 cell line treated with Ps-PR (FIG. 18). Here, the early effect would be evidenced in cell cycle progression. DNA damaged cells will accumulate in gap₁ (G₁), DNA synthesis (S), or in gap₂/mitosis (G₂/M) phase. In contrast, cells carrying irreversible damages to their genetic content will endure apoptosis, giving rise to the formation of fragmented DNA, which would be defined in subG₁ phase. The same amount of cell population in subG₁ phase in control and Ps-PR treated cells clearly proved the absence of apoptosis. In the control group, the main percentage of cell population was observed in G₁ phase, whereas in Ps-PR treated cells, a decrease in G₁ population was detected. In addition, the population of cells in both S and G₂/M phases in the treated cells are higher than the control one confirming the increase in the growth and division stages for treated cells (see Table 1).

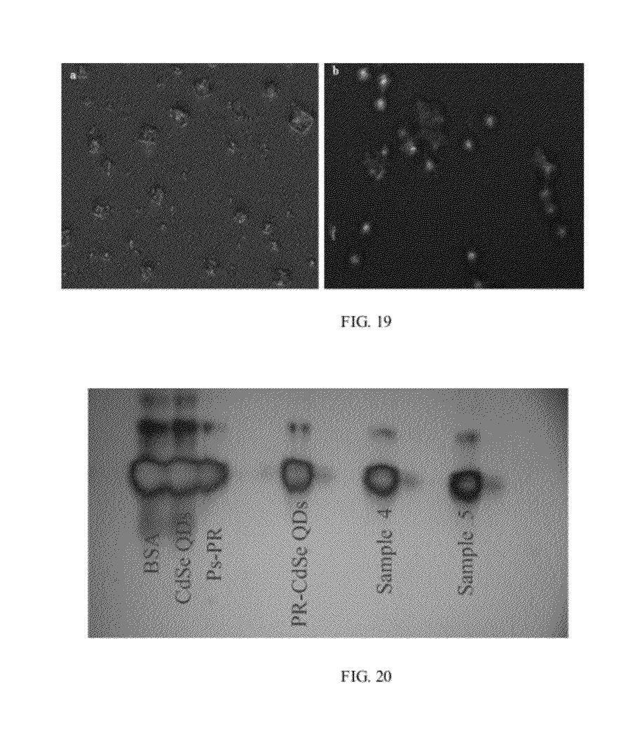

In order to examine the ability of hybrid nanostructures as anticancer drug delivery systems, DOX molecules (an anthracyclinic antibiotic) in two DOX/PR-Cys-CdSe QDs ratios were conjugated on their functional groups and subjected to the endocytosis and release inside the cancer cells. The MTT assay showed a good toxicity for anticancer drug delivery systems, DOX-PR-Cys-CdSe QDs, against L929 cell line. The toxicity of drug delivery systems strongly depends on the incubation time so that a considerable toxicity could be observed after 16 h of incubation. This behavior could be assigned to the either slow transferring from cell membrane or slow release of drug in the cell. In order to evaluate the first supposition, fluorescence microscopy (FIG. 19) was used to observe the rate of transferring of drug delivery systems from cell membrane. It was found that the rate of transfer of CdSe QDs through the cell membrane increase upon conjugation to Ps-PR. After 1 h incubation, PR-Cys-CdSe QDs transferred from the cell membrane completely while CdSe QDs were still transferring from the cell membrane even after 3 h; therefore some factors inside the cells retard the killing of the cancer cells. It seems the drug delivery systems are stable enough to escape the cytoplasm and insert the cell metabolism. The drugs release after disassociation of the self-assemblies and break down to their individual molecules by the cells.

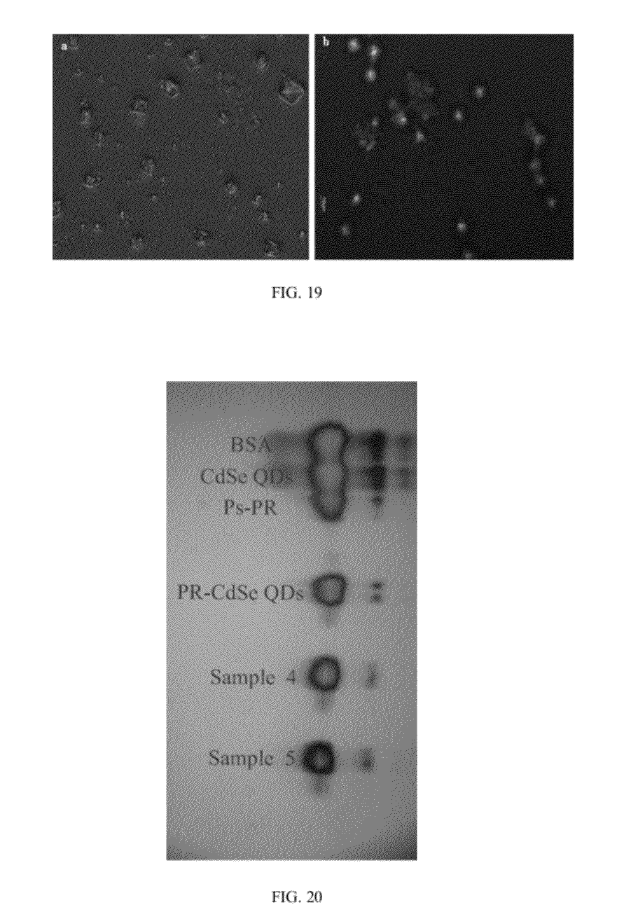

Understanding of the interactions between hybrid nanostructures and proteins is very important. In a biological fluid, proteins can be adsorbed or associated on nanoparticles. This adsorption can have significant impacts on biological, biochemical and cellular behavior. In order to check this absorption and the obtained protein conformational changes caused by this interaction, the interaction of the human transferrin with the synthesized samples via native gel electrophoresis was probed, then it was found that the human transferrin show a good tendency to attach to all samples; interestingly, no conformational changes on protein structure were observed. In addition, Ps-PR as the backbone of drug delivery systems and one of the main blocks in the molecular self-assemblies had lower tendency to absorb protein (FIG. 20).

This invention in its broader aspects and applications is not limited to the above embodiment and also directed to a large number of hybrid nanomaterials that may be formed from various pseudorotaxanes, pseudopolyrotaxanes, Rotaxanes, Polyrotaxanes, nanoparticles and Quantum Dots using different methods and reactions.

### EXAMPLES

The present invention will be further showed by the following examples, wherein the scope of the present invention is by no means limited by these Examples.

### Materials

Cadmium Chloride (CdCl₂ 2.5 H₂O), selenium powder (purity>99%), sodium hydroxide

(NaOH), mercaptoacetic acid, L-cysteine and sodium sulfite (Na₂SO₃), were purchased from Aldrich and used without further purification. Cyclodextrin (α and β) was provided from Fluka and dried prior use. Polyethylene glycol (MW=1000), cyanuric chloride (1, 3, 5-trichloro-2,4,6-triazin), dichloromethane, diethyl ether, silver nitrate, folic acid, Cisplatin [cis-dichlorodiammineplatinum (II), CDDP], N-hydroxysuccinimide (NHS) and 1-ethyl-3-3(3-dimethylaminopropyl) carbodiimide (EDC) were purchased from Merck. DMF was purchased from Merck and distilled from CaH₂. The cell lines (mouse tissue connective fibroblast adhesive cells (L929) were obtained from the National Cell Bank of Iran (NCBI) Pasteur institute, Tehran, Iran. 3-(4,5-Dimethylthiazol-2-yl)-2,5-diphenyltetrazolium bromide (MTT) powder, Annexin-V FLUOS Staining Kit, was obtained from Sigma. RPMI 1640 modified medium, fetal bovine serum (FBS) and penicillin/streptomycin solution were obtained from Gibco Invitrogen (Carlsbad, Calif.). Phosphoric acid used as the mobile phase in high-performance liquid chromatography (HPLC) was purchased from Merck. Deionized water was used in all experiments.

### Instruments

The Transmission Electron Microscopy (TEM) images were obtained using a LEO 912AB electron microscope with accelerating voltage of 200 kV. A Shimadzu UV-visible 1650 PC spectrophotometer was used for recording absorption spectra in solution using a cell of 1.0 cm path length Infrared (IR) spectra were recorded by a Nikolt 320 FT-IR. An ultrasonic bath (Model: SRS, 22 KHZ, Made in Italy) was used to disperse materials in solvents. ¹H NMR spectra were recorded in DMSO-d6 and D2O solvent on a bruker DRX 400 (400 MHz) apparatus with the solvent proton signal for reference.

Zeta potential and dynamic light scattering (DLS) diagrams were obtained using a Malvern-zs 20.4. A Varian Cary Eclipse fluorescence spectrophotometer was used for recording emission spectra in solution using a cell of 1.0 cm path length. Morphology and structure investigations were performed using the Philips XL30 scanning electron microscope (SEM) with 12 and 15 Accelerating voltages. The samples used for SEM observations were coated with a thin layer of gold. High-resolution surface imaging studies were performed using atomic force microscopy (AFM) to estimate surface morphology and particle size distribution. The samples were imaged with the aid of Dualscope/Rasterscope C26, DME, Denmark, using DS 95-50-E scanner with vertical z-axis resolution of 0.1 nm.

Photoluminescence (PL) emission spectra were recorded using a VARIAN Carey Eclipse fluorescence spectrometer. Images of solutions were recorded using a canon digital camera. Fluorescence images were recorded using a Trinocular inverted microscope bright field and phase contrast motic Spain model: AE31. Excitation of samples to record photographs or luminescence spectra was done as below:

CD/MAA-CdSe QDs at 415 nm, PR-CD/MAA-CdSe QDs at 420 nm, CDDP-PR-CD/MAA-CdSe QDs at 412 nm, FA-CDDP-PR-CD/MAA-CdSe QDs at 415 nm, Cys-CdSe QDs at 414 nm, PR-Cys-CdSe QDs at 434 nm and DOX-PR-Cys-CdSe QDs at 453, 557 and 597 nm.

A simple and reproducible reversed-phase high performance liquid chromatography (HPLC) with a Knauer liquid chromatograph (Smart line; Knauer, Berlin, Germany) equipped with an ultraviolet detector (Wellchrom, K-2600; Knauer) and a reverse-phase C18 column (Nucleosil H.P. 25 cm×0.46 cm internal diameter, pore size mm; Knauer) using isocratic elution with UV absorbance detection was developed and validated for determination of cisplatin in CDDP-PR-CD/MAA-CdSe QDs. The mobile phase was 15 mM phosphoric acid solution and flow rate was 1.00 mL/minute. The column effluent was detected at 210 nm. The retention time of free CDDP peak appeared between 2-4 minutes and the run time was 15 minutes. Linear regression with an acceptable linear relationship between response (peak area) and concentration in the range of 1 to 64 μg/mL was observed. The regression coefficient was 0.9999 and the linear regression equation was Y=34324X+15334. Sample concentrations were calculated using the calibration curves.

### Example 1

### Production of Cyclodextrin-Polyrotaxane End-Capped by Quantum Dots With Cysteine Capping Agent (PR-Cys-CdSe QDs)

Typically a solution of poly(ethylene glycol) (MW=1000), PEG, (9 g, 9×10⁻³ mol) and sodium hydroxide (0.64 g, 16×10⁻³ mol, in 5 ml water) was added dropwise to a solution of cyanuric chloride (13 g, 7×10⁻² mol, in 150 ml dichloromethane) and stirred at 0-40° C. for 1 h and then refluxed for 6 h. The mixture was then filtered and solvent was evaporated and obtained solid compound was dissolved in diethyl ether. The solution was filtered and precipitated in an ice bath. The precipitate was dissolved in dichloromethane, filtered and solvent was evaporated to obtain functionalized polyethylene glycol (Cl-PEG-C1) as colorless oil [39].

Then Cl-PEG-Cl (1 gr, 0.77 mmol) was dissolved in 2 ml distilled water and added to a reaction flask containing a suspension of α-CD in distilled water (3.75 gr, 3.85 mmol, in 2 ml distilled water) with vigorous stirring at 25° C. The mixture of reaction was stirred at room temperature for 3 h. After reaction, the obtained mixture was filtered and the precipitate was washed with water to remove the excess α-CD and functionalized PEG. The pseudopolyrotaxane (Ps-PR) was obtained as a white powder after drying by vacuum oven at 40° C. ¹H NMR (400 MHz, DMSO-d₆) δ 3.50-3.58 (H-4 and H-2 of α-cyclodextrin), 3.77-3.92 (H-6-3-5 of α-cyclodextrin and CH₂CH₂ of PEG), 4.90 (H-1 (anomeric proton) of α-cyclodextrin). IR (cm⁻¹, KBr): 1029 (C—OH), 1153 (C—O—C), 1701 (C═N), 2927(C—H), 3371 (O—H).

Finally, cyclodextrin-polyrotaxane end-capped by quantum dots with cysteine capping agent (PR-Cys-CdSe QDs), as a hybrid nanomaterials, was prepared as following procedure. Typically about 0.05 gr quantum dots with cysteine capping agent (Cys-CdSe QD), see below, was dissolved in 10 ml distilled water, then 0.1 gr pseudopolyrotaxane was added to the solution of reaction and the obtained mixture left in an ultrasonic bath for 5 minutes. The mixture of reaction was stirred at room temperature for at least 72 h. Then the obtained mixture was filtered and the solvent was then evaporated under reduced pressure. The sample was then dissolved in distilled water (5 ml) and dialyzed against water (1 h) to give PR-Cys-CdSe QDs as a yellow powder.

¹H NMR (400 MHz, D₂O) δ 2.9-3.2 (CH₂ and CH of L-cysteine capping agent), 3.6-5 (H-4-2-6-3-5-1 of α-cyclodextrin and CH₂CH₂ of PEG) IR (cm⁻¹, KBr): 1031 (C—OH), 1151 (C—O—C), 1400 (symmetric CO₂), 1587(asymmetric CO₂), 2923 (C—H), 3344 (O—H overlapping NH₂).

Cys-CdSe QD was prepared as follows:

Cysteine (0.5 g, 4.13 mmol) was added to a solution of CdCl₂2.5H₂O (0.6840 g, 3.4 mmol) in distilled water (50 ml) at 90° C. under constant stirring and the pH of the solution was adjusted to 10 with NaOH (1 M). Afterward, a water solution of Na₂SeSO₃ (0.1 M, 20 ml) was injected into the reaction flask at 80° C. under high-intensity ultrasonic. The mixture was ultrasonicated for additional 30 minutes and then quantum dots were separated from solution by addition of acetone and centrifugation. ¹H NMR (400 MHz, D₂O) δ 2.7-3.3 (CH₂ and CH of L-cysteine capping agent). IR (cm⁻¹, KBr): 600-800 (C—S), 1398 (symmetric CO₂), 1579 (asymmetric CO₂), 3384(NH₂).

### Example 2

Production of cyclodextrin-polyrotaxane end-capped by quantum dots with beta-cyclodextrin (β-CD) and mercaptoacetic acid (MAA) capping agents (PR-CD/MAA-CdSe QDs): PR-CD/MAA-CdSe QDs was prepared in the same manner as explained in Example 1 except that quantum dots with beta-cyclodextrin and mercaptoacetic acid capping agents (CD/MAA-CdSe QDs), see below for the preparation strategy, was used instead of the Cys-CdSe QD. ¹H NMR (400 MHz, D₂O) δ 2.75-5 (Protons both CD/MAA-CdSe QDs and Ps-PR). IR (cm⁻¹, KBr): 1000-1300 (asymmetric glycosidic vibrations of pseudopolyrotaxane backbone), 1581.52 (CO₂), 2952 (C—H), 3375 (OH).

CD/MAA-CdSe QDs was prepared as follows:

For preparation of CdSe QDs containing both MAA and CD capping agents, CdCl₂.H₂O (0.6840 g, 3.4 mmol) was dissolved in 50 ml distilled water at room temperature. Upon addition of MAA (0.3 ml, 4.31 mmol) to this solution, white colloids appeared. Then HS-β-CD [40] (0.025 g, 2 mmol) was added to this mixture and dispersed in the reaction mixture by stirring at room temperature. pH was brought to 11 by addition of NaOH (1 M) solution. Then the mixture was placed in an ultrasonic bath at 80° C. for 15 minutes and water solution of Na₂SeSO₃ (0.1 M, 20 ml), made by refluxing Na₂SO₃ (0.63 g, 5.00 mmol) and selenium powder (0.2 g, 2.50 mmol) in 50 ml of water for 3 h under N₂ atmosphere, was added to the reaction mixture. Mixture was left in ultrasonic bath for 30 minutes to obtain a yellow solution. The solution was stirred and heated at 90° C. under N₂ atmosphere for 1 h, then it was cooled to room temperature and product was separated upon precipitation in acetone and then centrifugation. Pure CD/MAA-CdSe QDs was obtained as a fine crystalline yellow compound after drying in vacuum oven. ¹H NMR (400 MHz, D₂O) δ 3.22-3.37 (Protons both β-CD and MAA capping agents). IR (cm⁻¹, KBr): 1385 (CH₂), 1575 (CO₂), 2925 (C—H), 3440 (OH).

As mentioned before, hybrid nanomaterials are promising candidates in order to use in variety of applications. For example to prove the efficacy of the molecular self-assemblies as drug delivery systems, folic acid (FA), doxorubicin (DOX) and cisplatin (Cis-Diamminedichloroplatinum (CDDP) a platinum-based chemotherapy drug) were conjugated to their functional groups of hybrid nanomaterials of Examples 1 and 2. These compounds were prepared as follows:

### Example 3

### Conjugation of DOX to PR-Cys-CdSe QDs (DOX-PR-Cys-CdSe QDs)

EDC (0.0004 g, 0.002 mmol), NHS (0.00023 g, 0.002 mmol) and DOX (0.0015 g, 0.0027 mmol) were added to a 100 ml 3-neck round-bottom flask containing 5 ml distilled water and pH of solution was adjusted at 7.4 and mixture was stirred at room temperature for 30 minutes. Then a solution of PR-Cys-CdSe QDs (0.01 g in 20 ml distilled water) was added to above mixture at 25° C. The mixture was stirred for 6 h at 25° C. and then dialyzed against water (1 h) to obtain the final product. IR (cm⁻¹, KBr): 1031 (C—OH), 1151 (C—O—C), 1647(amide bond), 2923 (C—H), 3344 (O—H).

### Example 4

### Conjugation of CDDP to PR-CD/MAA-CdSe QDs (CDDP-PR-CD/MAA-CdSe QDs)

For conjugating of CDDP to PR-CD/MAA-CdSe QDs, CDDP must form aqueous complexes firstly similar to a reported procedure in the literature [41]. CDDP (10 mg, 0.033 mmol) was dissolved in 10 ml distilled water then 10 mg AgNO₃ (0.059 mmol) was added to the reaction mixture. The mixture was stirred at room temperature in the dark for at least 12 h. After reaction the obtained mixture was centrifuged to eliminate the AgCl precipitate which was produced during the reaction as it proceeded. Then the supernatant was filtered to obtain purified solution.

In the next step 0.05 g PR-CD/MAA-CdSe QDs was dissolved in 5 ml distilled water and added to the above solution. The obtained solution was stirred at room temperature for 72 h in the dark to form CDDP complexes with PR-CD/MAA-CdSe QDs. The resulting solution was filtered and dialyzed to obtain pure product as a clear yellow solution. IR (cm⁻¹, KBr): 1618.17 (CO₂ overlapping NH bending vibrations).

### Example 5

### Conjugation of FA to CDDP-PR-CD/MAA-CdSe QDs (FA-CDDP-PR-CD/MAA-CdSe QDs)

Carboxyl functional groups of FA molecules can be coupled to the free hydroxyl groups of cyclodextrin molecules of CDDP-PR-CD/MAA-CdSe QDs. For this purpose folic acid must be activated firstly by ester formation between it and NHS molecules by using an EDC coupling reagent. In this method briefly, folic acid (0.004 g, 0.009 mmol) was dispersed in 20 ml distilled water, and then NHS (0.001 g 0.009 mmol) and EDC (0.0017 g, 0.009 mmol) were added to the mixture. The mixture stirred in the dark at room temperature for 12 h. The resulting mixture was centrifuged and the obtained precipitate was added to the aqueous solution of CDDP-PR-CD/MAA-CdSe QDs. The obtained mixture stirred at room temperature in the dark for at least 12 h. After reaction the resulting yellow-orange clear solution was dialyzed to obtain pure product. IR (cm⁻¹, KBr): 1382 (C—N and C—O), 1569 (NH), 1604 (C═C of folic acid), 1699.17 (CO₂ overlapping C═N of folic acid).

### REFERENCES

[1]. Harada, A.; Hashidzume, A.; Yamaguchi, H.; Takashima, Y. Chem. Rev. 2009, 109, 5974-6023.
[2]. Yin, J.; Dasgupta, S.; Wu, J. Org. Lett. 2010, 12, 1712-1715.
[3]. Fang, L.; Olson, M. A.; Benitez, D.; Tkatchouk, E.; Goddart, W. A.; Stoddart, J. F. Chem. Soc. Rev. 2010, 39, 17-29.
[4]. Wenz, G.; Han, B-H.; Muller, A. Chem. Rev. 2006, 106, 782-817.
[5]. Maniam, S.; M. Cieslinski, M.; F. Lincoln, S.; Onagi, H.; J. Steel, P.; C. Willis, A.; J. Easton, C. Org. Lett. 2008, 10, 1885-1888.
[6]. Okada, M.; Harada, A. Org. Lett. 2004, 6, 361-364.
[7]. Gutsche, C. D. Calixarenes; Monographs in Supermolecular Chemistry; The Royal Society of Chemistry: Cambridge, U.K., 1989; Vol. 1.
[8]. Calixarenes: A Versatile Class of Macrocyclic Compounds; Vicens, J., Böhmoer, V., Eds.; Topics in Inclusion Science; Kluwer Academic Publishers: Dordrecht, The Netherland, 1991; Vol. 3.
[9]. Smith, B. H. Bridged Aromatic Compounds; Organic Chemistry; Academic Press: New York, 1964; Vol. 2.
[10]. Amabilino, D. B.; Ashton, P. R.; Balzani, V.; Boyd, S. E.; Credi, A.; Lee, J. Y.; Menzer, S.; Stoddart, J. F.; Venturi, M.; Williams, D. J. J. Am. Chem. Soc. 1998, 120, 4295.
[11]. Reymo, F. M.; Stoddart, J. F. Chem. Rev. 1999, 99, 1643.
[12]. Ogino, H. J. Am. Chem. Soc. 1981, 103, 1303.
[13]. Isnin, R.; Kaifer, A. E. J. Am. Chem. Soc. 1992, 114, 3136.
[14]. Harada, A.; Li, J.; Kamachi, M. J. Chem. Soc., Chem. Commun. 1997, 1413.
[15]. Whang, D.; Park, K.-M.; Heo, J.; Kim, K. J. Am. Chem. Soc. 1998, 120, 4899.
[16]. Kim, K. Chem. Soc. Rev. 2002, 31, 96.
[17]. Bissell, R. A.; Cordova, E.; Kaifer, A. E.; Stoddart, J. F. Nature (London) 1994, 369, 133.
[18]. Okada, M.; Harada, A. Macromolecules 2003, 36, 9701-9703.
[19]. Aubin-Tam, M. E.; Hwang, W.; Hamad-Schifferli, K. Proc. Natl. Acad. Sci. U.S.A 2009, 106, 4095-4100.
[20]. Aubin-Tam, M. E.; Hamad-Schifferli, K. Langmuir 2005, 21, 12080-12084.
[21]. Jensen, G. J.; Kornberg, R. D. Proc. Natl. Acad. Sci. U.S.A 1998, 95, 9262-9267.
[22]. Zanchet, D.; Micheel, C. M.; Parak, W. J.; Gerion, D.; Alivisatos, A. P. Nano Lett. 2001, 1, 32-35.
[23]. Xiao, Y.; Patolsky, F.; Katz, E.; Hainfeld, J. F.; Willner, I. Science 2003, 299, 1877-1881.
[24]. J. Ackerson, C.; D. Jadzinsky, P.; Z. Sexton, J.; A. Bushnell, D.; D. Kornberg, R. Bioconjugate Chem. 2010, 21, 214-218.
[25]. Nam, T.; Park, S.; Lee, S—Y.; Park, K.; Choi, K.; Song, I C.; Han, M H.; J. Leary, J.; Andrew Yuk, S.; Chan Kwon, I.; Kim, K.; Young Jeong, S. Bioconjugate Chem. 2010, 21, 578-582.
[26]. Querner, C.; Reiss, P.; Zagorska, M.; Renault, O.; Payerne, R.; Genoud, F. J Mater Chem 2005, 15, 554-563.
[27]. Coe, S.; Woo, WK.; Bawendi, V.; Bulovic, M. Nature 2002, 420, 800-803.
[28]. Tessler, N.; Medvedev, V.; Kazes, M.; Kan, S H.; Banin, U. Science 2002, 295, 1506-1508.
[29]. Fafard, S.; Hinzer, K.; Raymond, S.; Dion, M.; McCaffrey, J.; Feng, Y. Science 1996, 274, 1350-1353.
[30]. Klimov, V I.; Mikhailovsky, A A.; Xu, S.; Malko, A.; Hollingsworth, J A.; Leatherdale, C A. Science 2000, 290, 314-317.
[31]. Medintz, I L.; Clapp, A R.; Mattoussi, H.; Goldman, E R.; Fisher, B.; Mauro, J M. Nat Mater 2003, 2, 630-638.
[32]. Ruedas-Rama, M J.; Wang, X J.; Hall, E A H. Chem Commun 2007, 15, 1544-1546.
[33]. Lin, C A J.; Liedl, T.; Sperling, R A.; Fernandez-Arguelles, M T.; Costa-Fernandez, J M.; Pereiro, R. J Mater Chem 2007, 17, 1343-1346.
[34]. Han, M Y.; Gao, X H.; Su, J Z.; Nie, S, Nat Biotechnol. 2001, 19, 631-635.
[35]. Jaiswal, J K.; Mattoussi, H.; Mauro, J M.; Simon, S M. Nat. Biotechnol. 2003, 21, 47-51.
[36]. Medintz, I L.; Uyeda, H T.; Goldman, E R.; Mattoussi, H. Nat Mater 2005, 4, 435-446.
[37]. Mondejar, S P.; Kovtun, A.; Epple, M. J Mater Chem 2007, 17, 4153-4159.
[38]. Janczewski, D.; Tomczak, N.; Khin, Y W.; Han, M-Y.; Vancso, G. J. European Polymer Journal 2009, 45, 3-9.
[39]. Namazi, H.; Adeli, M. Polymer 2005, 46, 10788.
[40]. Benkhaled, A.; Cheradame, H.; Fichet, O.; Teyssié, D.; Buchmann, W.; Guégan, P. Carbohydrate polymers 2008, 73, 482.
[41]. Ndinguri, M.; Solipuram, R.; Gambrell, R.; Aggarwal, S.; Hammer, R. Bioconjugate Chem. 2009, 20, 1869-1878.

We claim:

1. A hybrid nanomaterial comprising two or more building blocks selected from the group consisting of a rotaxane, a polyrotaxane, a pseudorotaxane, a pseudopolyrotaxane, a quantum dot, a polymer, a nanoparticle and any combination thereof.

2. The hybrid nanomaterial according to claim 1, comprising one quantum dot and at least one other building block selected from the group consisting of polyrotaxane, rotaxane, pseudopolyrotaxane and pseudorotaxane.

3. The hybrid nanomaterial according to claim 1, comprising one nanoparticle and at least one other building block selected from the group consisting of polyrotaxane, rotaxane, pseudopolyrotaxane and pseudorotaxane.

4. The hybrid nanomaterial according to claim 1, wherein the building blocks are connected via covalent interactions or non-covalent interactions or any combination thereof.

5. The hybrid nanomaterial according to claim 5, wherein said non-covalent interaction comprises host-guest interaction, hydrogen bond, van der Waals interaction, electrostatic interaction, dispersion interaction, or any combination thereof.

6. The hybrid nanoparticle according to claim 1, comprising shapes selected from the group consisting of core-shell, spindle, spindle-like and necklace.

7. The hybrid nanomaterial according to claim 1, further comprising an end-capping agent.

8. The hybrid nanomaterial according to claim 1, wherein the rotaxane, polyrotaxane, pseudorotaxane, and/or pseudopolyrotaxane comprises cyclodextrin.

9. The hybrid nanoparticle according to claim 1, wherein the building block comprises a polymer containing a biocompatible carbohydrate backbone, wherein the biocompatible carbohydrate backbone comprises polyethylene glycol.

10. The hybrid nanoparticle according to claim 1, comprising:
a cyclodextrin-polyrotaxane end-capped by quantum dots with a cysteine-comprising capping agent, a cyclodextrin-polyrotaxane end-capped by cadmium selenide quantum dots;
a cyclodextrin-polyrotaxane end-capped by quantum dots with a beta-cyclodextrin or mercaptoacetic acid capping agent or combinations thereof;
a cyclodextrin-polyrotaxane end-capped quantum dot having covalent interactions between pseudopolyrotaxane and cadmium selenide quantum dots, with a cysteine end-capping agent; or
a cyclodextrin-polyrotaxane end-capped quantum dot having non-covalent interactions between pseudopolyrotaxane and cadmium selenide quantum dots with a beta-cyclodextrin or a mercaptoacetic acid end-capping agents or combinations thereof.

11. The hybrid nanoparticle according to claim 1, wherein the hybrid nanoparticle is conjugated with an active compound.

12. The hybrid nanoparticle according to claim 1, wherein the hybrid nanoparticle is conjugated with an active compound selected from the group consisting of an antibiotic, doxorubicin, cis-diamminedichloroplatinum, and folic acid.

13. A drug delivery or drug targeting system comprising the hybrid nanomaterial according to claim 1 conjugated with an active compound, in particular a drug.

14. A diagnostic system comprising the hybrid nanomaterial according to claim 1.

15. A nanocomposite comprising the hybrid nanomaterial according to claim 1.

16. A biomolecular or cellular imaging system comprising the hybrid nanomaterial according to claim 1.

17. A method for the synthesis of a hybrid nanomaterial comprising the steps of conjugating a cysteine-cadmium comprising quantum dot through a nucleophylic reaction between functional amino groups with end functional groups of a (pseudo)polyrotaxane.

18. The method according to claim 17, further comprising conjugating carboxylate groups of the quantum dot with a drug to obtain a drug delivery system.

19. The method according to claim 18, wherein the drug comprises a prophylactic agent against malaria, an antibiotic or an anticancer drug, wherein the drug comprises doxorubicin or cis-diamminedichloroplatinum.

20. A method for delivering an active compound or drug to a cell comprising providing a hybrid nanomaterial according to claim 11 comprising contacting a cell with the drug- or active compound-comprising hybrid nanomaterial.
